(19) United States
(12) Patent Application Publication
(10) Pub. No.: US 2010/0074396 A1
(43) Pub. Date: Mar. 25, 2010

(54) MEDICAL IMAGING WITH BLACK SILICON PHOTODETECTOR

(75) Inventors: Matthias J. Schmand, Lenoir City, TN (US); Debora Henseler, Erlangen (DE); Ronald Grazioso, Knoxville, TN (US); Nan Zhang, Knoxville, TN (US); Mark S. Andreaco, Knoxville, TN (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Medical Solutions USA, Inc., Malvern, PA (US)

(21) Appl. No.: 12/498,101

(22) Filed: Jul. 6, 2009

Related U.S. Application Data
(60) Provisional application No. 61/078,494, filed on Jul. 7, 2008.

(51) Int. Cl. A61B 6/03; G01T 1/24; G01T 1/20

(52) U.S. Cl. 378/19; 250/370.09; 250/370.11

(57) ABSTRACT

Medical imaging may be accomplished with a high photoconductive gain at a relatively low operating voltage by employing a black silicon photodetector and integrating CMOS components with elements of the photodetector.

### PRIORITY CLAIM

This application claims priority from a U.S. Provisional titled “Black-Silicon Based Detector For X-ray and Gamma-ray Imaging” having U.S. Ser. No. 61/078,494, the entire contents of which is herein incorporated by reference.

### TECHNICAL FIELD

The present disclosure relates to medical imaging devices and methods using black silicon photodetectors.

### BACKGROUND

State-of-the-art X-ray and gamma ray imaging modalities typically use detectors based on a combination of a scintillator and a photodetector. The scintillator converts high-energy radiation into visible light, then the photodetector converts the visible photons into an electrical signal, which usually is amplified by front-end readout electronics.

Two main nuclear medicine modalities are positron emission tomography (PET) and single-photon emission computed tomography (SPECT). Commercial PET and SPECT detectors typically use an inorganic scintillator material in combination with a photomultiplier tube and pulse-counting readout electronics. In recent years, detectors based on semiconductor detectors such as silicon PIN diodes, silicon drift diodes, or avalanche diodes (APDs) have become available and are the subject of current development activities in industry and academia.

X-ray computed tomography (CT) systems commonly use detectors containing scintillator material and silicon PIN diodes. Charge-integrating front-end electronics produce detector signals that are proportional to total charge during a given read-out interval.

In all these imaging modalities, the signal-to-noise ratio depends critically on the conversion efficiency of the scintillator, the quantum efficiency (QE) for detecting the visible photons, and the noise of the read-out electronics. High signal levels can be obtained by use of avalanche photodiode and photo-multiplier tube detectors. The intrinsic gain of these detectors provides beneficial signal-to-noise ratios, which may be aided by integration of a first amplification stage of the read-out electronics at a location very close to the detector. However, avalanche photodiodes and photo-multiplier tubes require extreme drive voltages in excess of 300 Volts (V). The high voltages impose the need for special drive circuitry. The overall system is thus burdened with high cost and complexity. In addition, the direct integration of CMOS read-out circuitry on the photodiode wafer is not feasible when these high drive voltages are needed.

A need therefore exists for improved medical imaging devices and methods with higher resolution detectors that operate with lower drive voltages.

### SUMMARY

The above needs are fulfilled, at least in part, by receiving high energy radiation from a patient body, i.e., gamma rays emitted from the patient body, such as for computed tomography, or X-rays transmitted through the patient body, such as for PET and SPECT, converting the received radiation into visible light, exposing the visible light to a black silicon photodetector to produce an electrical signal, and generating an image of the patient body from the electrical signal. The high energy radiation may be directed to scintillator pixels, which may be in registration with pixel locations on the black silicon photodetector. In addition, the black silicon photodetector may include a plurality of sub-pixels for each scintillator pixel. The detector may further include CMOS structures integrated with the black silicon photodetector pixel locations. A low reverse bias voltage, for example about 3 Volts, may be applied to the photodetector for detection of the radiation.

The above needs are further fulfilled by a medical imaging device, which includes a high-energy radiation source, such as X-rays or gamma rays, a scintillator, a black silicon photodetector optically coupled to the scintillator, and a read-out circuit coupled to the black silicon photodetector. The black silicon photodetector may include pixel locations, which may be in registration with pixels of the scintillator. The black silicon photodetector may further include subpixels for each pixel of the scintillator. The read-out circuit may further include CMOS components integrated with the black silicon photodetector pixel locations. The black silicon photodetector may include a silicon wafer substrate with black silicon photodiodes and the CMOS components integrated on the silicon wafer substrate. Alternatively, the CMOS structures may be vertically integrated with the black silicon photodetector pixels. The black silicon photodetector elements may be formed on a first surface of the silicon wafer, a second silicon wafer may be bonded to a second surface of the first silicon wafer, and the CMOS components may be formed on the second silicon wafer. In addition, a wavelength shifting layer may be located between the scintillator and the black silicon photodetector. With the integration of the CMOS components with the black silicon photodiodes on the silicon wafer, digitization of the electrical signal from the detector may be performed on the silicon wafer to generate an image of the patient.

Additional aspects and technical effects of the present disclosure will become readily apparent to those skilled in the art from the following detailed description wherein embodiments of the present disclosure are described simply by way of illustration of the best mode contemplated to carry out the present disclosure. As will be realized, the present disclosure is capable of other and different embodiments, and its several details are capable of modifications in various obvious respects, all without departing from the present disclosure. Accordingly, the drawings and description are to be regarded as illustrative in nature, and not as restrictive.

### BRIEF DESCRIPTION OF THE DRAWINGS

The present disclosure is illustrated by way of example, and not by way of limitation, in the figures of the accompanying drawing and in which like reference numerals refer to similar elements and in which:

FIG. 1 illustrates peak-like silicon microstructures at the surface of black silicon;

FIG. 2 schematically illustrates interband states in black silicon;

FIG. 3 illustrates a medical imaging device including a pixellated scintillator and a pixellated black silicon photodetector, in accordance with exemplary embodiments of the present disclosure;

FIG. 4 illustrates a medical imaging device including a pixellated scintillator and a black silicon photodetector with subpixels, in accordance with exemplary embodiments of the present disclosure;

FIG. 5 illustrates a medical imaging device including a monolithic scintillator and a pixellated black silicon photodetector, in accordance with exemplary embodiments of the present disclosure;

FIG. 6 illustrates a medical imaging device including a pixellated scintillator and a monolithic black silicon photodetector, in accordance with exemplary embodiments of the present disclosure;

FIGS. 7A-7C illustrate medical imaging devices including a pixellated scintillator and a pixellated black silicon photodetector with integrated CMOS structures, in accordance with exemplary embodiments of the present disclosure;

FIG. 8 illustrates a medical imaging device including a pixellated scintillator, a pixellated black silicon photodetector, and a wavelength shifting layer, in accordance with exemplary embodiments of the present disclosure;

FIG. 9 illustrates a medical imaging device including a scintillator with subpixels, a pixellated black silicon photodetector, and a wavelength shifting layer, in accordance with exemplary embodiments of the present disclosure;

FIG. 10 illustrates a medical imaging device including a highly pixellated scintillator and a black silicon photodetector with subpixels, in accordance with exemplary embodiments of the present disclosure; and

FIG. 11 illustrates a medical imaging device including a scintillator with virtual subpixels and a black silicon photodetector with subpixels, in accordance with exemplary embodiments of the present disclosure.

### DETAILED DESCRIPTION

In the following description, for the purposes of explanation, numerous specific details are set forth in order to provide a thorough understanding of exemplary embodiments. It should be apparent, however, that exemplary embodiments may be practiced without these specific details or with an equivalent arrangement. In other instances, well-known structures and devices are shown in block diagram form in order to avoid unnecessarily obscuring exemplary embodiments.

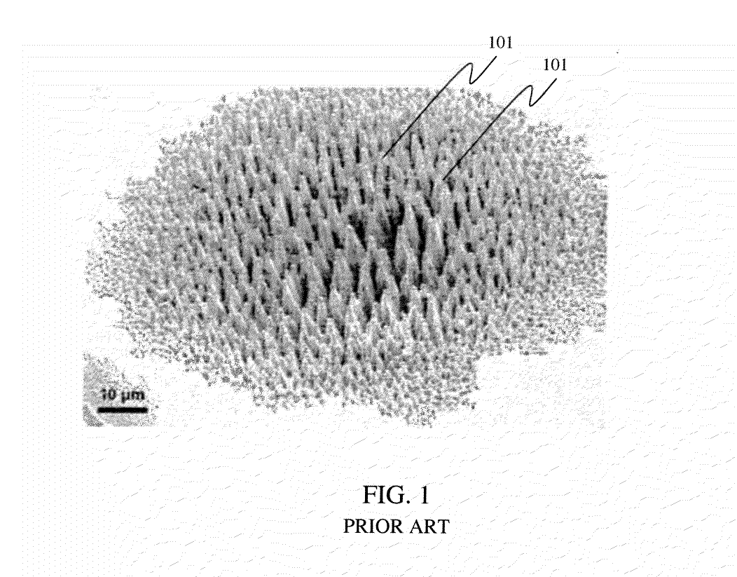

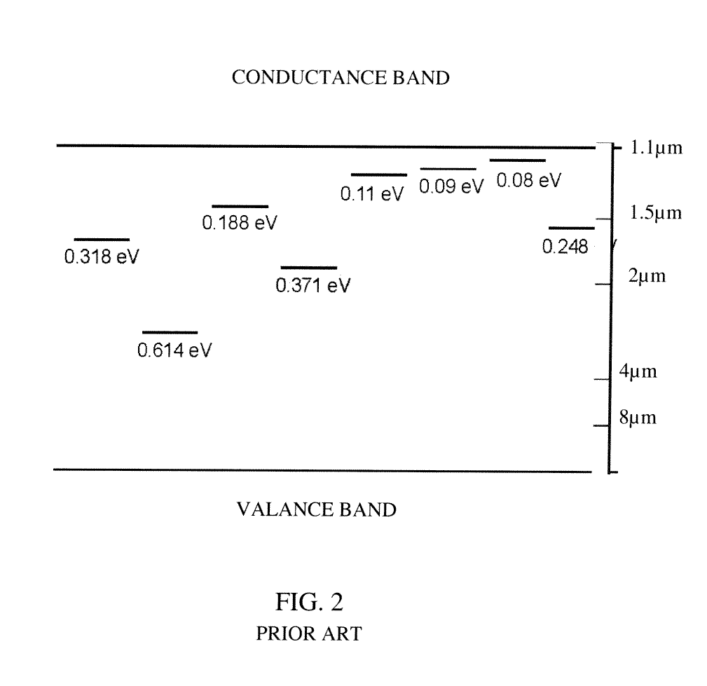

Black silicon refers to a modified silicon surface layer, where a standard silicon wafer surface is turned into a black absorber material by treatment with femtosecond (fs) laser pulses in the presence of a sulfur-containing gas such as sulfur hexafluoride (SF₆) or hydrogen sulfide (H₂S) (or by incorporating other dopants, e.g., Oxygen (0), Selenium (Se), or Tellurium (Te)). Similar surface modifications by wet-chemical etching or plasma etching are also known. The results of black-silicon formation by fs laser irradiation are the formation of peak-like silicon microstructures at the surface, as illustrated at 101 in FIG. 1, and/or the formation of interband states in the silicon, as illustrated in FIG. 2. The surface modifications lead to a highly improved absorption of the silicon surface layer over the whole visible range. Improvement in absorption of the black silicon structure is particularly large in the red and infrared wavelength regions in comparison with untreated silicon which is a rather poor absorber with absorption lengths of several microns (μm) up to several millimeters (mm).

The described surface modification also leads to the formation of a n/n+ heterojunction between the bulk crystalline silicon and the modified black silicon layer. Applying a reverse bias voltage to this junction via suitable contacts leads to a photodetector device, which has the additional advantage of photoconductive gain which can be as high as 1200 at only a 3V reverse bias. The photoconductive gain is related to the formation of interband states by the doping. This photoconductive gain yields a photosensor with a large responsivity and high signal-to-noise ratio.

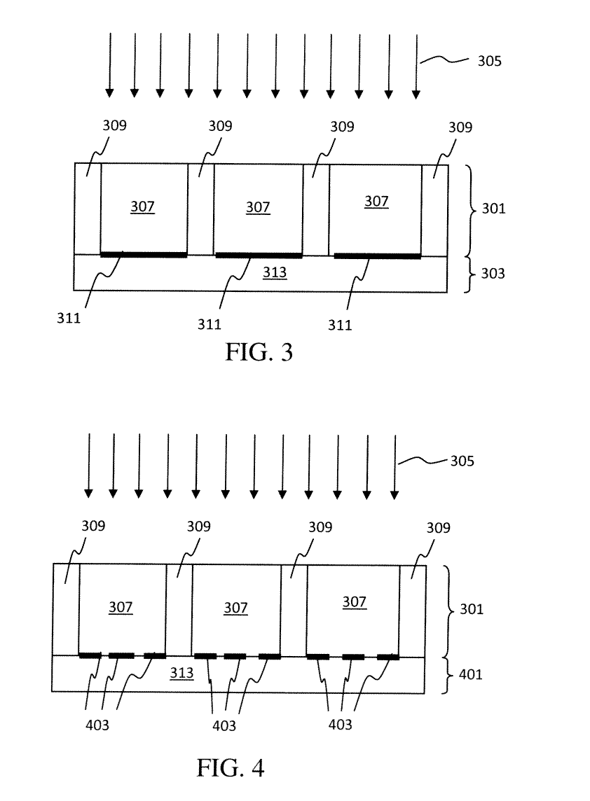

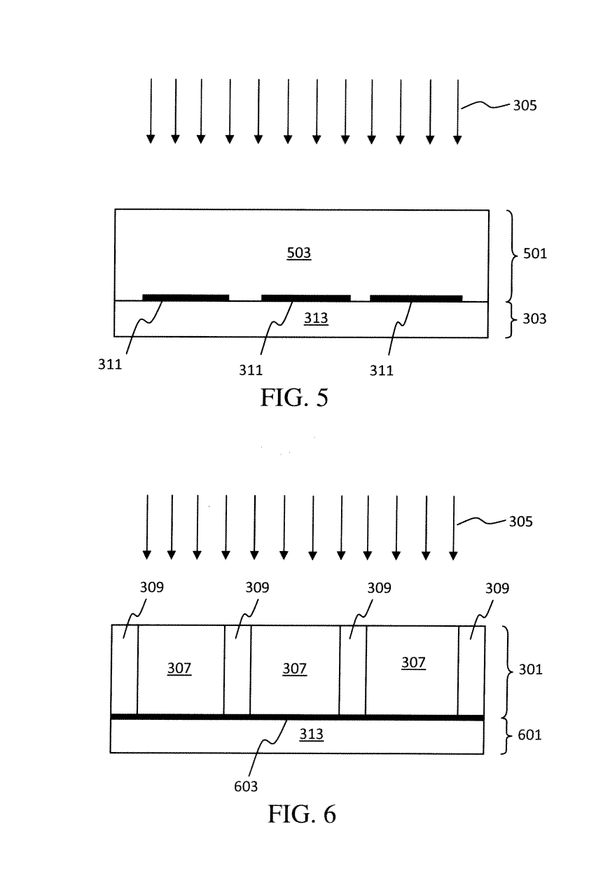

Adverting to FIG. 3, a radiation detector for X-ray and gamma ray medical imaging applications is shown. As illustrated, scintillator 301 in combination with black-silicon based photodetector 303 measure incident high-energy (i.e., X-ray or gamma) radiation 305. Scintillator 301 is shown formed of scintillator elements 307, separated by septa 309. Photodetector 303 is formed of black silicon elements 311 in wafer substrate 313. In FIG. 3, both scintillator 301 and photodetector 303 are pixellated with the same pixel pitch and are in registration with each other. However, as illustrated in FIGS. 4-6, respectively, it is also possible to have different pixel numbers for the scintillator and the detector (FIG. 4), to optically couple a monolithic scintillator to a pixellated photodetector (FIG. 5), or to use a pixellated scintillator block together with a monolithic photodetector (FIG. 6).

As illustrated in FIG. 4, photodetector 303 may be replaced with photodetector 401, in which n (shown with n equal to three) black silicon sub-elements 401 are aligned with each scintillator element 307. Sub-elements 403 form subpixels which are smaller than the pixels used for obtaining the spatial resolution of an image. Such a design has advantages for the count-rate capacity of the detector, because the count rates are then limited by the number of times a subpixel is hit by an X or gamma quantum, and the counts per subpixel are a factor of n smaller. Alternatively, scintillator elements 307 may be further divided into sub-elements.

In FIG. 5, scintillator 501 is substituted for scintillator 301. Scintillator 501 is formed of a monolithic slab optically coupled to the pixellated photodetector 303. Similarly, FIG. 6 illlustrates an exemplary embodiment in which a monolithic photodetector 601, formed of a black silicon slab 603, is coupled to pixellated scintillator 301.

In FIGS. 3, 4, and 6, the scintillator need not be structured mechanically in the form of pixilation, but may be pixellated by virtual scintillation cells (or pixels) within a monolithic scintillator slab. The virtual optical cells may be created by laser scribing or post growth processing, such as forging. The virtual cell guides the scintillation light in a preferred direction, preventing light from spreading through the slab.

Electrical contacts are not shown in any of FIGS. 3 through 6. However, a suitable metallization is provided to contact each black silicon element 311 in FIGS. 3 and 5 (or each sub-element 403 in FIG. 4 or black silicon slab 603 in FIG. 6) and the bulk silicon on the other side of a junction between the bulk silicon wafer substrate 303 and the black silicon element 311 (or each element 403 in FIG. 4 or black silicon slab 603 in FIG. 6). The metal contacts may be provided to the bulk silicon either separately for each pixel or as a common contact to the bulk silicon layer. The contacts may then be routed to the side of the silicon wafer by metallization lines and/or to the back of the wafer by via holes, where they may be bonded to the first part of the read-out electronics for further amplification and signal processing.

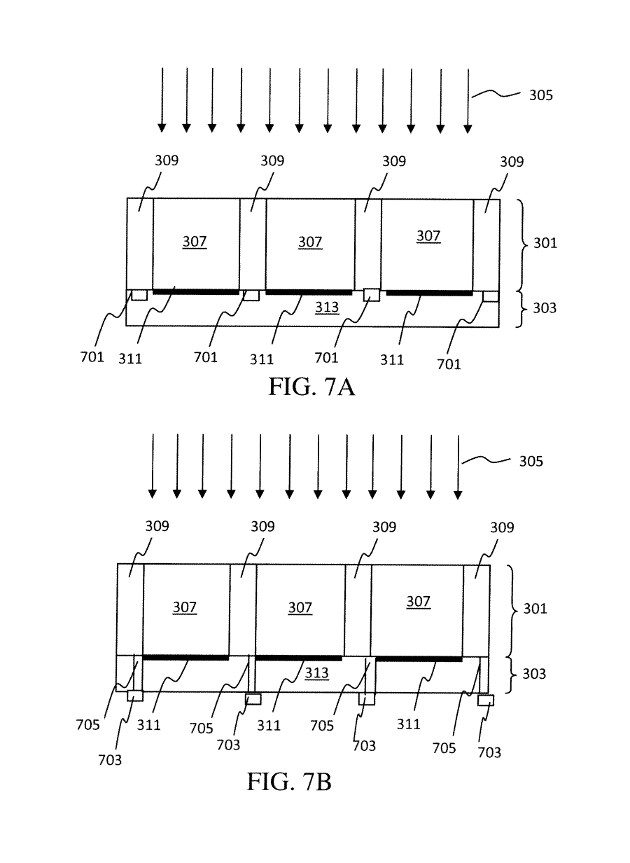

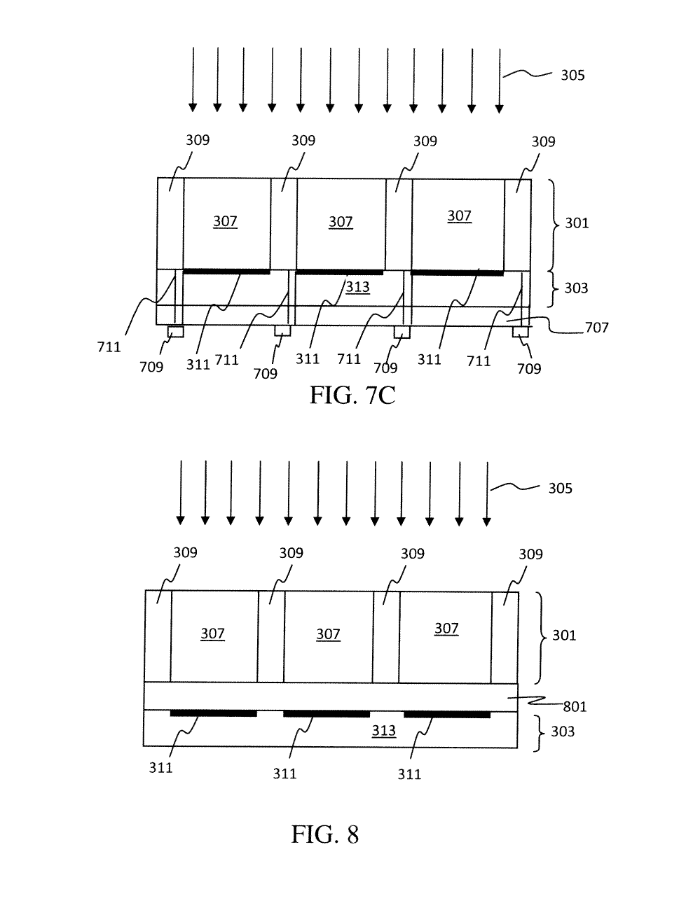

In another exemplary embodiment, the black silicon diode pixels may be monolithically integrated with the first part of the read-out electronics on the same wafer. As schematically illustrated in FIG. 7A, CMOS components 701 are integrated on the same side of the same silicon substrate as black silicon elements 311. Alternatively, the CMOS structures may be integrated underneath the black silicon junction or buried in deeper layers of the silicon wafer. Such vertical integration (3D detector) may be accomplished either by joining separate wafers or by depositing further epi-layers of silicon on top of the CMOS structures to form the diode junction. FIG. 7B illustrates CMOS components 703 formed on the opposite surface of silicon substrate 303, connected to black silicon elements 311 through vias 705 and metallization or contacts (not shown for illustrative convenience) from the vias to the black silicon elements 311. Although the CMOS structures are shown under the septa 309, they may alternatively be formed directly under the black silicon elements. FIG. 7C illustrates a configuration in which a second wafer 707 is bonded to the lower surface of silicon substrate 303 by wafer bonding, and CMOS structures 709 are formed on the lower surface of the second wafer 707. The CMOS structures are connected to the black silicon elements 311 through vias 711 and metallization or contacts (not shown for illustrative convenience) from the vias to the black silicon elements. The CMOS structures may alternatively be formed in between the silicon wafers.

Returning briefly to FIG. 4, each subpixel may also be connected to its own CMOS electronics components (e.g., a comparator and a counter), and the signal for each macropixel may be obtained by processing the subpixel contributions digitally. This detector example is particularly suitable for high count-rate applications such as counting CT or a combined, counting PET/CT or SPECT/CT detector. A design in which the scintillator has a much finer sub-pixel structure may also be used, e.g., by using a scintillator which grows in wave-guiding, needle-like microstructures such as cesium iodide (CsI). Then there may be many scintillator needles coupled to each black-silicon detector pixel (or sub-pixel).

Integration of the CMOS structures with the black silicon elements is possible because the manufacturing methods for the black silicon layer are compatible with state-of-the-art CMOS processes, and the low bias voltages (e.g., 3V) are compatible with CMOS wafer voltage ranges. Such an active pixel device layout is particularly beneficial for applications such as CT, where there are often many hundred small pixels (about 1 mm² or smaller) integrated in one detector module. Integration of the CMOS structures with the photodetector elements allows the digitization of the analog detector response to be performed on the substrate itself without need of requiring further electronics. Components integrated in the CMOS parts of the wafer may, for example, include a preamplifier, signal shaper, analog-to-digital converter, comparator, and/or pulse counter.

As illustrated in FIG. 8, for the detection of blue scintillation light (such as the 420 nm cerium doped lutetium oxyorthosilicate (LSO) emission currently used in PET detectors or the 410 nm sodium iodide (Nal) emission used in SPECT cameras), it may be beneficial to use a wavelength shifting layer 801 with high conversion efficiency in between the scintillator 301 and the black silicon detector 303 to shift the light from blue to green, red, or even infrared emission, where the quantum efficiency of the black-silicon diode is high. Although shown in FIG. 8 with a scintillator and black silicon detector such as those in the embodiment of FIG. 3, wavelength shifting layer 801 may be employed between the scintillator and black silicon detector in any of the embodiments of FIGS. 3 through 7. The wavelength shifting layer may itself be structured into pixels or sub-pixels by optically separating elements such as septa, air gaps or internal interfaces.

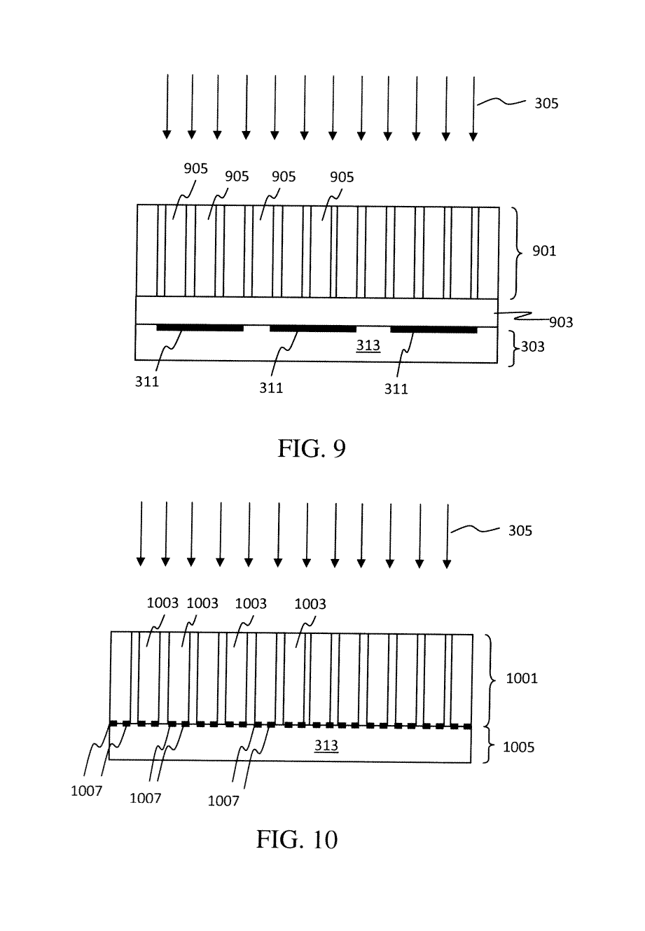

Adverting to FIG. 9, a PET or SPECT detector is illustrated with a pixellated scintillator block 901, a wavelength shifting layer 903, which also acts as a light guide to mix the spatial profile for each crystal emission, and an array of black silicon detectors 311 to detect the red-shifted light. In this exemplary embodiment, the number of black silicon diodes 311 is smaller than the number of scintillator crystals 905, as the gamma-ray position is obtained by using the Anger principle and pixel position look-up tables. It should be noted that the wavelength shifting and the light mixing functionalities of wavelength shifting layer 903 may be split between two different optical layers sandwiched on to of each other.

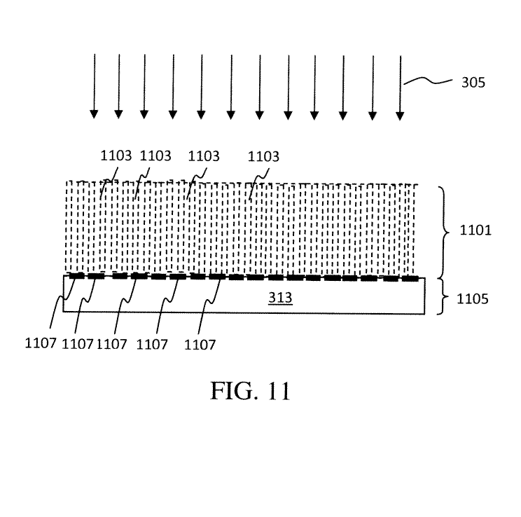

Two further exemplary embodiments are illustrated in FIGS. 10 and 11. FIG. 10 includes a highly pixelized scintillator array 1001 with scintillator elements 1003, whereas FIG. 11 includes a scintillator slab 1101 with virtual optical cells 1103, coupled. In FIG. 10, scintillator 1001 is coupled to a monolithic black silicon 3D detector 1005, and in FIG. 11, scintillator 1101 is coupled to monolithic black silicon 3D detector 1105. In FIG. 10, the number of diode elements is higher than the number of scintillator elements (or sub-pixels). This configuration allows over-sampling the black silicon diodes while still allowing clear identification of the impinged scintillator element, in case of optical cross talk between scintillator elements, and provides sub-pixels for each scintillator element allowing high photon flux counting capability. The configuration illustrated in FIG. 11 has a higher number of scintillator elements or virtual cells than black silicon elements. The virtual crystals may also be described as (but not limited to) sub millimeter fiber bundles or needle like crystal structures, which provide directed optical pathways within the scintillator. The spatial resolution of such a detector will be dominated by the black silicon diode dimension.

Embodiments of the present disclosure, using a black silicon photodetector in a scintillator based detector module to measure X-rays and gamma rays for medical imaging, have several advantages which address different needs for Angiography, Fluoroscopy, Radiographic Systems, CT, PET, SPECT, or combined PET/CT, SPECT/CT, or PET/SPECT detectors. Specifically, the disclosed embodiments can achieve several technical effects, including providing a high absorption of visible photons, due to a high quantum efficiency of the detector and, in turn, to good quantum statistics and improved energy resolution in a pulse-counting detector or, alternatively, an improved signal-to-noise ratio in a charge-integrating detector. Also, a photoconductive gain of the order of several hundred or thousand can be achieved, which improves the signal-to-noise level by amplifying the signal even before the actual read-out circuit and reduces the needed level of further amplification. In addition, the gain is achieved at low bias voltages (in contrast to avalanche photodiodes), which enables a compatibility with CMOS structures on the same wafer. This can give rise to highly integrated, active pixel designs, which are particularly suitable for high count-rate applications, using very small sub-pixel sizes and a digital processing of the sub-pixel outputs to yield an overall pixel signal. The present disclosure enjoys industrial applicability in various medical imaging devices.

In the preceding description, the present disclosure is described with reference to specifically exemplary embodiments thereof. It will, however, be evident that various modifications and changes may be made thereto without departing from the broader spirit and scope of the present disclosure, as set forth in the claims. The specification and drawings are, accordingly, to be regarded as illustrative and not as restrictive. It is understood that the present disclosure is capable of using various other combinations and embodiments and is capable of any changes or modifications within the scope of the inventive concept as expressed herein.

What is claimed is:

1. A medical imaging method comprising:
receiving high-energy radiation from a patient body;
converting the received radiation into visible light;
exposing the visible light to a black silicon photodetector to produce an electrical signal; and
generating an image of the patient body from the electrical signal.

2. The medical imaging method according to claim 1, wherein the step of receiving comprises receiving gamma rays emitted from the patient body or X-rays transmitted through the patient body.

3. The medical imaging method according to claim 2, wherein the step of receiving comprises directing the high-energy radiation to scintillator pixels.

4. The medical imaging method according to claim 3, wherein the step of exposing comprises directing the visible light from the scintillator pixels to pixel locations on the black silicon photodetector in registration therewith.

5. The medical imaging method according to claim 4, wherein the black silicon photodetector comprises a plurality of sub-pixels for each scintillator pixel.

6. The medical imaging method according to claim 4, wherein CMOS components are integrated with the black silicon photodetector pixel locations.

7. The medical imaging method according to claim 6, wherein the step of exposing further comprises applying a low reverse bias voltage to the black silicon photodetector pixel locations.

8. The medical imaging method according to claim 7, wherein the reverse bias is approximately 3 Volts.

9. A medical imaging device comprising:
a high-energy radiation source;
a scintillator;
a black silicon photodetector optically coupled to the scintillator; and
a read-out circuit coupled to the black silicon photodetector.

10. The medical imaging device according to claim 9, wherein the high-energy radiation source comprises X-rays or gamma rays.

11. The medical imaging device according to claim 10, wherein the black silicon photodetector comprises pixel locations.

12. The medical imaging device according to claim 11, wherein the black silicon photodetector pixel locations are in registration with pixels of the scintillator.

13. The medical imaging device according to claim 12, wherein the black silicon photodetector comprises a plurality of sub-pixels for each pixel of the scintillator.

14. The medical imaging device according to claim 11, wherein the read-out circuit comprises CMOS components integrated with the black silicon photodetector pixel locations.

15. The medical imaging device according to claim 14, wherein the black silicon photodetector further comprises a silicon wafer substrate, and the black silicon photodetector pixel locations and the CMOS components are integrated on the silicon wafer substrate.

16. The medical imaging device according to claim 14, wherein the CMOS components are vertically integrated with the black silicon photodetector pixel locations.

17. The medical imaging device according to claim 16, wherein the black silicon photodetector further comprises:
a first silicon wafer substrate having a first surface adjacent the scintillator and a second surface opposite the first surface, the black silicon elements being formed on the first surface of the first silicon wafer; and
a second silicon wafer bonded to the second surface, wherein the CMOS components are formed on the second silicon wafer.

18. The medical imaging device according to claim 13, further comprising a wavelength shifting layer located between the scintillator and the black silicon photodetector.

19. The medical imaging device according to claim 18, wherein the read-out circuit comprises CMOS components vertically integrated with the black silicon photodetector pixel locations.

20. A medical imaging method for X-ray computed tomography comprising:
transmitting X-rays from a radiation source through a body of a patient;
applying a reverse bias voltage of about 3 Volts;
sensing the transmitted radiation by a detector comprising:
a pixellated scintillator, the scintillator converting the radiation into visible light;
a pixellated photodetector, the photodetector converting the photons into an electrical signal, the photodetector comprising a silicon wafer and black silicon photodiodes formed on the silicon wafer; and
CMOS components integrated with the black silicon photodiodes on the silicon wafer;
digitizing the electrical signal from the detector on the silicon wafer; and
generating an image of the patient body.
